(19) United States
(12) Patent Application Publication
(10) Pub. No.: US 2018/0221682 A1
(43) Pub. Date: Aug. 9, 2018

(54) PHOTOTHERAPY APPARATUS FOR HAIR AND SKIN TREATMENT

(71) Applicant: Apira Science, Inc., Irvine, CA (US)

(72) Inventors: Morgan Pepitone, Irvine, CA (US); Jeffrey Braile, Irvine, CA (US)

(21) Appl. No.: 15/948,605

(22) Filed: Apr. 9, 2018

Related U.S. Application Data
(63) Continuation-in-part of application No. 15/204,184, filed on Jul. 7, 2016, now abandoned.
Application No. 15/204,184 is a continuation-in-part of application No. 14/286,061, filed on May 23, 2014, now abandoned.
(60) Provisional application No. 61/826,687, filed on May 23, 2013.

(51) Int. Cl. A61N 5/06

(52) U.S. Cl. CPC A61N 5/0617; A61N 2005/007; A61N 2005/0663; A61N 2005/0647

(57) ABSTRACT

A wearable hands-free apparatus for providing phototherapy treatment for a number of facial skin and scalp conditions, as well as promotion of hair growth. The apparatus includes a base platform component that is structured to be worn on a user's head, such as an adjustably sizable headband, earphones or eyeglasses. The base platform includes a pivoting attachment member to allow selective interchanging of different phototherapy face masks for particular facial skin treatments, as well as selective interchanging of different head canopies for various scalp and hair growth treatments. The face masks and head canopies each include a light emitting plate fitted to an inner side that faces the user and having an array of light generating sources, such as light emitting diodes (LEDs), laser diodes, or infrared lights that emit light within a particular wavelength corresponding with the particular phototherapy treatment.

This patent application is a Continuation-In-Part of pending patent application Ser. No. 15/204,184 filed on Jul. 7, 2016 which is a Continuation-In-Part of pending U.S. patent application Ser. No. 14/286,061 filed on May 23, 2014 which is based on provisional patent application Ser. No. 61/826,687 filed on May 23, 2013.

### BACKGROUND OF THE INVENTION

### Field of the Invention

This invention relates to light therapy for the treatment of various skin, hair and scalp conditions, and more particularly, to a hands-free phototherapy apparatus with interchangeable light emitting face masks and head canopies for providing evenly distributed light of various beneficial wavelengths that is directed onto either a user's facial skin or onto the user's scalp for providing a variety of phototherapy treatments.

### Discussion of the Related Art

People are frequently confronted with hair loss, as well as a variety of different scalp and skin-related conditions, such as acne, sun spots, and wrinkling of the skin, psoriasis and non-melanoma skin cancer. In response, an assortment of treatment products, each typically targeting one specific hair, scalp or skin-related condition, have been developed over the past 75 years and made available to the public. Many of these products are in the form of a topical solution that requires an arduous application process.

Scientists throughout the years have determined that there are beneficial effects of various wavelengths of light directed onto the skin, and that absorption of light is the key to cellular change. Light therapy emits photons which are absorbed by the skins photoreceptors. Hair and skin cells respond well to phototherapy involving low level light due to the fact that cells reside just underneath the skin surface, making these low levels of energy able to reach the receptor sites and induce photochemistry.

Phototherapy consists of exposure to specific wavelengths of light using lasers, light emitting diodes (LED's) (both individual and arrays), IPL's (Intense Pulsed Light) and other light sources, for a prescribed amount of time to both treat disease and affect cosmetic enhancements to the hair, scalp and skin, particularly facial skin. The use of phototherapy in medical science and cosmetic services is rapidly evolving as more and more wavelengths of light are being identified to target various sections of cells in order to stimulate cellular proficiency and enhance the body's ability to heal and rejuvenate itself. Phototherapy is currently used to treat acne, wrinkles, sun and age spots, rosacia, eczema, hair loss and wound healing through wavelengths indicated by various colors (i.e., wavelengths) of the light spectrum. By utilizing various wavelengths, colors relatively close on the spectrum can cause different effects when applied to various parts on the body.

Red and infrared lights have been used to increase the production of collagen and to reduce redness, dilated capillaries and damage to the skin, as well as reduction of wrinkles and fine lines. Blue light has been clinically shown to reduce acne and, when combined with red light, eliminates acne and reduces the scarring often associated with acne treatment. Yellow and Amber lights have been clinically shown to reduce fine lines and wrinkles, rosacia, and can help to repair sun damaged skin. Green light has been shown to reduce and eliminate sun and age spots, lighten freckles and also help promote more luminous skin condition and overall radiance of the skin.

There are a number of phototherapy devices currently available for home use to treat both skin and hair. The majority of these are hand held devices, varying in both size and number of light sources (i.e., laser diodes, LED's, or infrared diodes). These devices are manually moved around the hair or face by the user and require a constant movement in order to expose the entire surface area to the light sources. This results in an uneven treatment protocol, as the average user is unlikely to be able to cover the entire surface area through manual movements and will leave certain areas untreated. Further, due to the need for a manageable size (must fit in the hand), these devices are often underpowered.

Several phototherapy devices have been developed that are adapted to be portably worn by a user in a hands-free mode of operation. For example, U.S. Pat. App. Pub. No. 2009/0012586 A1 to Kepecs discloses a system that houses LEDs within a head unit that resembles a baseball helmet. The Kepecs device is used for reducing hair loss, as well as the therapeutic healing of a variety of skin disorders. One particular shortcoming of the Kepecs device is the onerous task of snapping or screwing in different LEDs to alter the desired wavelength.

U.S. Pat. App. Pub. No. 2006/0030908 to Powell et al. discloses a skin treatment phototherapy device that may comprise a clamshell structure, pen shape, facial mask, or desk lamp design, and which includes multi-colored LEDs. The Powell device attempts to treat a variety of skin conditions on the face and other skin regions below the user's head. Depending on the skin condition to be treated, the corresponding wavelengths, intensity levels, and time interval for the skin treatment can be varied by a control system.

The various phototherapy devices in the related art fail to provide for the ability to quickly and conveniently interchange different light emitting plate members to a hands-free headgear to allow for treatment of various skin, hair and scalp conditions that require different light wavelengths. Moreover, the phototherapy devices in the prior art do not allow for easy pivotal adjustment of a light emitting face plate towards and away from a user's facial skin and also pivotal adjustment of a head canopy towards and away from the user's scalp, and wherein different light emitting face plates and light emitting head canopies can be easily interchanged as needed for various types of treatments.

### Objects and Advantages of the Invention

Considering the foregoing, it is a primary object of the present invention to provide a wearable hands-free phototherapy apparatus for providing light therapy for treatment of various facial skin conditions, scalp conditions, and promotion of hair growth, and wherein the phototherapy apparatus includes a base platform component worn on the user's head and one or more interchangeable light emitting face masks and one or more interchangeable light emitting head canopies that removably attach to the base platform component with the use of a pivoting attachment member on the base platform component.

It is a further object of the present invention to provide a phototherapy apparatus including an adjustably sizable headband and a selection of interchangeable light emitting face masks and head canopies that are easily attached and removed from the base platform component for providing distributed light of various beneficial wavelengths to treat various facial skin and scalp conditions, as well as to promote hair growth.

It is still a further object of the present invention to provide a phototherapy apparatus for treatment of various facial skin conditions, hair growth promotion and scalp conditions and wherein the apparatus includes an adjustably sizable headband and one or more light emitting face masks and light emitting head canopies that can be selectively and easily attached and removed from the headband and which are adjustably positionable towards and away from the user's face or scalp while wearing the headband.

It is still a further object of the present invention to provide a phototherapy apparatus for treatment of various facial skin conditions, hair growth promotion and scalp conditions and wherein the apparatus includes an adjustably sizable headband that has a pivoting attachment member for removable and pivotable interchanged attachment of different light emitting face masks and head canopies.

It is yet a further object of the present invention to provide a phototherapy apparatus including a base platform structured to be worn on a user's head and having a pivoting attachment member on the front of the base platform that is adapted for easy and convenient interchanged attachment of various light emitting face masks and head canopies thereto, and wherein each face mask and head canopy, when attached to the headband, is easily adjusted towards and away from the user's head via the pivoting attachment member.

These and other objects and advantages of the present invention are more readily apparent with reference to the following detailed description and the accompanying drawings.

### SUMMARY OF THE INVENTION

The present invention is directed to a wearable hands-free apparatus for providing phototherapy treatment for a number of facial skin and scalp conditions, as well as promotion of hair growth. The apparatus includes a base platform component that is structured to be worn on a user's head, such as an adjustably sizable headband, earphones or eyeglasses. The base platform includes a pivoting attachment member to allow selective interchanging of different phototherapy face masks for particular facial skin treatments, as well as selective interchanging of different head canopies for various scalp and hair growth treatments. The face masks and head canopies each include a light emitting plate fitted to an inner side that faces the user. The light emitting plate of each of the face masks and head canopies has an array of light generating sources, such as light emitting diodes (LEDs), laser diodes, or infrared lights that emit light within a particular wavelength corresponding with the particular phototherapy treatment.

### BRIEF DESCRIPTION OF THE DRAWINGS

For a fuller understanding of the nature of the present invention, reference should be made to the following detailed description taken in conjunction with the accompanying drawings in which:

FIG. 1 is a perspective view of an adjustably sizable headband of the present invention showing a hinged attachment member on the front of the headband for interchangeable attachment and adjustable positioning of different phototherapy light emitting face masks and head canopies to the headband;

FIG. 2 is an isolated perspective view, shown in partial cross section, illustrating a foam padding on the headband and a headband side pivot for hinged attachment with a pivot component on the attachment member;

FIG. 3 is an inside perspective view of the phototherapy apparatus of the present invention showing an interchangeable phototherapy light emitting face mask fitted to the dovetail hinge member of the headband;

FIG. 4 is a right, rear perspective view showing the interchangeable phototherapy light emitting face mask separated above a dovetail hinge attachment member on the headband, demonstrating removable attachment thereto for allowing interchanging of different phototherapy light emitting face masks to the headband and adjustable positioning of the attached face mask towards and away from the user's face;

FIG. 5 is a left rear perspective view showing separation of the phototherapy light emitting face mask from the dovetail hinge member on the headband;

FIG. 6 is a bottom plan view of a head canopy showing a spaced arrangement of light generating sources with corresponding light openings in an inner reflective shell, along with an arrangement of spacer columns for engaging the user's head and a dovetail slot for removable attachment to the dovetail hinge member on the headband;

FIG. 7 is a side perspective view illustrating the manner of attachment and removal of the head canopy from the pivoting attachment member on the headband;

FIG. 8 is a side elevational view showing the headband and face plate operatively worn on a user with the face plate positioned in spaced relation from the user's face; and

FIG. 9 is a side elevational view showing the headband and head canopy operatively positioned on the user's head with the head canopy positioned in close spaced relation above the user's scalp/hair.

Like reference numerals refer to like parts throughout the several views of the drawings.

### DETAILED DESCRIPTION OF THE PREFERRED EMBODIMENTS

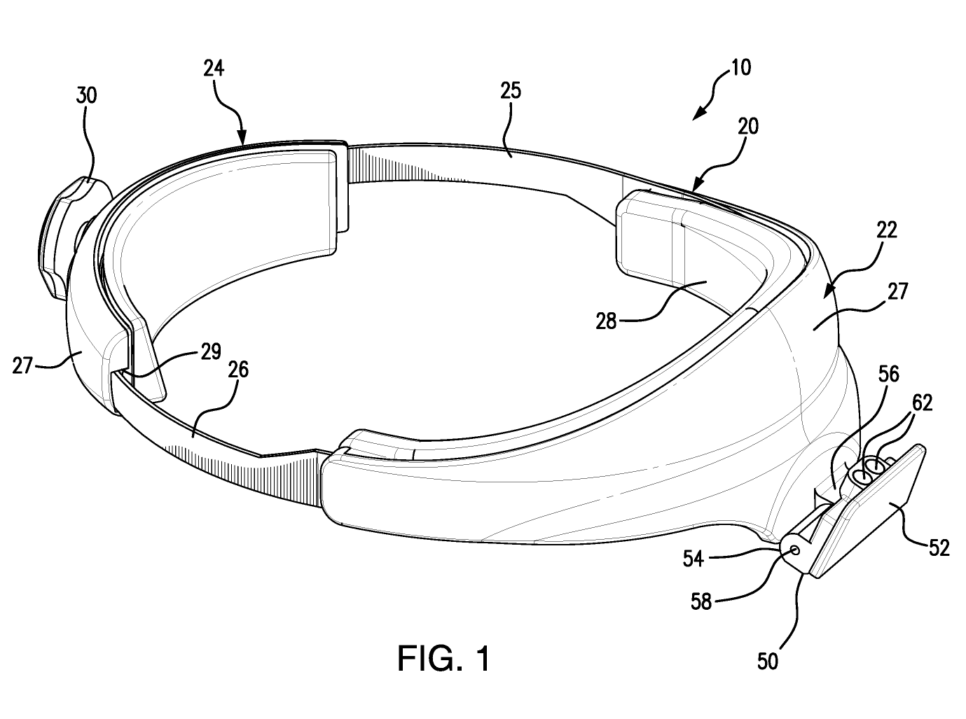

Referring to FIGS. 1-5, the wearable hands-free phototherapy apparatus is shown according to a preferred embodiment of the invention and is generally indicated as 10.

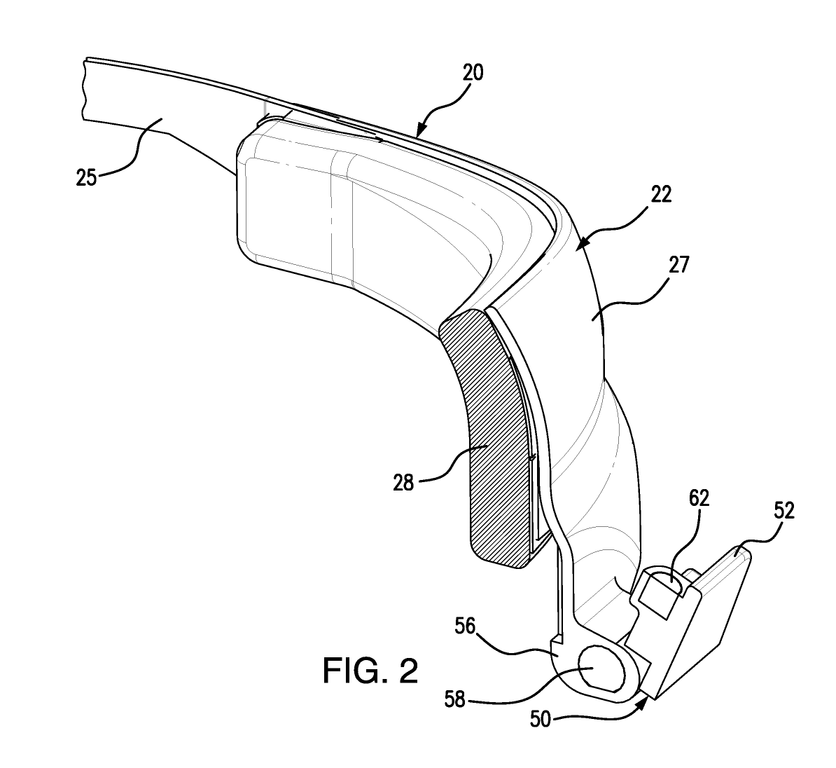

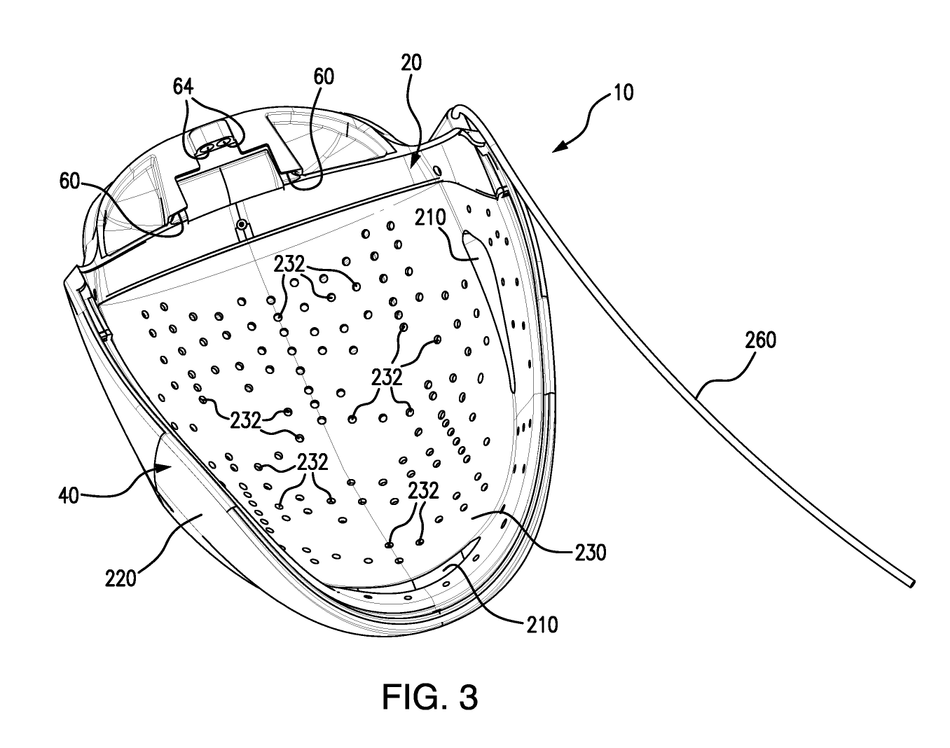

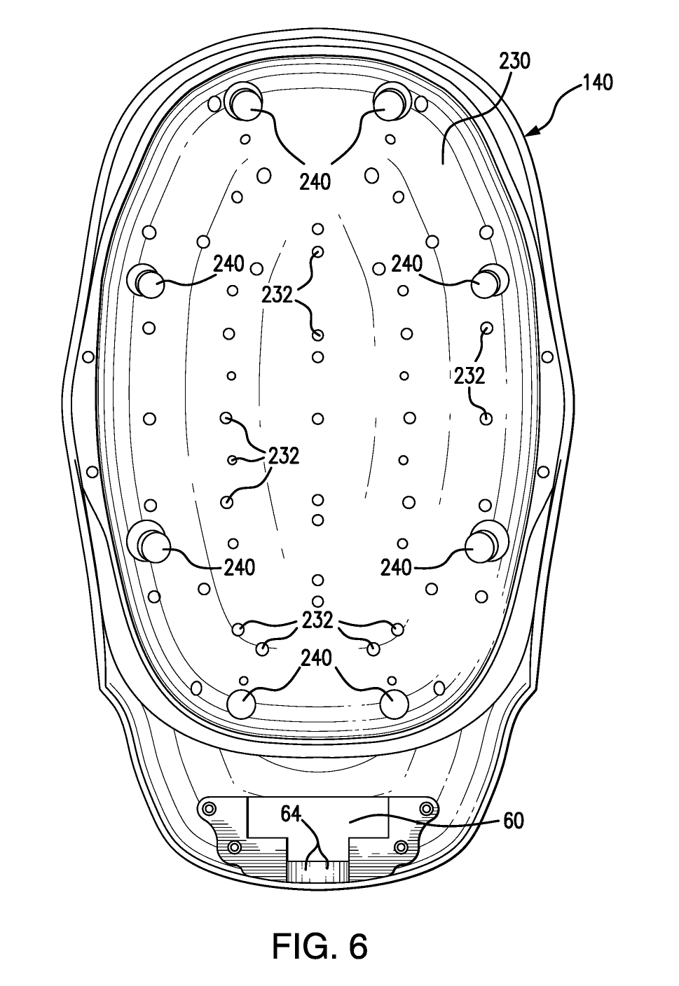

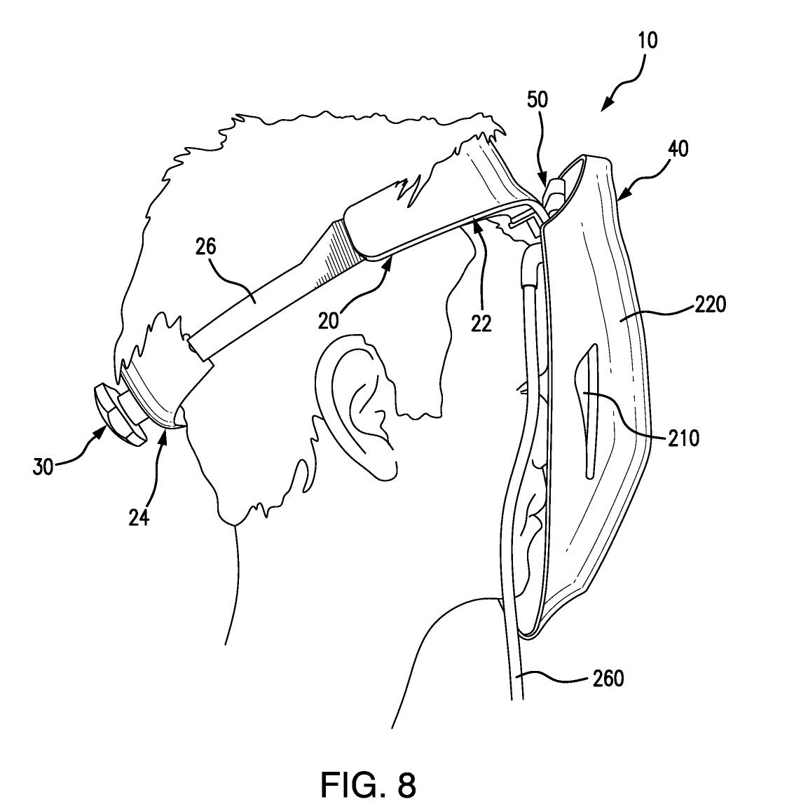

The phototherapy apparatus 10 includes a headband 20 and, in a preferred embodiment, multiple interchangeable face plates 40 (see FIGS. 3-5 and 8) and head canopies 140 (see FIGS. 6, 7 and 9), wherein each face plate 40 and head canopy 140 is specifically structured to emit light within a particular wavelength range correlating with one or more types of treatments of one or more facial skin conditions, scalp conditions or promotion of hair growth. For example, in treating inflammation, lesions, or canker sores, a range (628 nm-694 nm) of red wavelengths is preferable; in treating rosacea or wrinkling of the skin, a range (568 nm-590 nm) of yellow wavelengths is preferable; in treating acne, a range (405 nm-476 nm) of blue wavelengths is preferable; in treating age spots, sun damage, or hyperpigmentation, a range (514 nm-543 nm) of green wavelengths is preferable; and in stimulating the skin to produce collagen and elastin, a range (700 nm-1090 nm) of infrared wavelengths is preferable. As seen in FIGS. 1 and 2, the headband 20 includes a front portion 22 and a rear portion 24, and left and right flexible strap members 25, 26 extending from the front portion 22 and interconnecting with the rear portion 24. The front and rear portions each include an outer hard plastic shell 27 and a cushion material 28 on the inner side for engagement with the user's head, and particularly across the forehead and temple regions, as well as the back of the head. FIG. 2 shows a cross section of the cushion material 28 on the front portion 22 of the headband 20 that engages the forehead and temple areas of the head. The flexible strap members 25, 26 are fixed to the front portion 22 and extend into an interior slotted channel 29 within the rear portion 24 for adjusting the size of the headband to fit properly on the user's head. More particularly, the flexible strap members 25, 26 are able to be pulled into the slotted channel 29 or driven out of the slotted channel by rotation of an adjustment knob 30 on the rear portion 24 of the headband 20. Rotation of the adjustment knob 30 in one direction (e.g., clockwise) serves to pull the left and right flexible strap members 25, 26 into and through the slotted channel 29 of the rear portion of the headband to reduce the overall cranial circumference of the headband 20. In this manner, the headband 20 can be tightened to fit properly around the user's head. Rotation of the adjustment knob 30 in the opposite direction (e.g., counterclockwise) serves to urge the left and right flexible strap members 25, 26 out of the slotted channel 29 on each side of the rear portion of the headband, thereby enlarging the overall cranial circumference of the headband 20. This allows the size of the headband 20 to be effectively enlarged to properly fit to a particular wearer's head size.

In the preferred embodiment, as shown in FIGS. 1-5, the face plates 40 are each removable from the headband 20 to allow interchanging of different face plates. In order to allow for easy and quick removal and replacement of different face plates 40 on the headband 20, the headband is fitted with a pivoting dovetail hinge member 50 on the front portion 22 of the headband. As seen in FIGS. 1 and 2, the dovetail hinge member 50 includes a dovetail mounting member 52 and a dovetail side pivot 54 that is pivotally joined with a headband side pivot 56 on the front of the headband. A torque hinge 58 is fitted through both the dovetail side pivot 54 and the headband side pivot 56 to pivotally join the dovetail side pivot to the headband side pivot, allowing pivotal movement of the dovetail hinge member 50 through a range of motion relative to the headband 20, and particularly the front portion 22 of the headband 20. Each of the face plates 40 includes a dovetail slot 60 on the top inner facing side of the face plate for congruent, fitted receipt of the dovetail hinge mounting member 52 therein. As seen in FIGS. 3-5, the dovetail hinge mounting member 52 is received through the bottom of the dovetail slot 60 on the face plate 40. To attach the face plate 40 to the headband 20, the dovetail slot 60 of the face plate is positioned above the dovetail hinge member 50 and then lowered down onto the dovetail hinge mounting member 52, allowing the dovetail hinge mounting member 52 to be slidably received within the dovetail slot 60 until corresponding magnets 62, 64 on both the dovetail hinge member 50 and the face plate 40 engage and magnetically join to one another, thereby securing the face plate 40 on the headband 20. Once properly secured and fitted to the headband, the face plate 40 can be pivotally adjusted towards and away from the user's face by simply grasping the lower edge or side edges of the face plate and urging the face plate either towards or away from the user's face, while the phototherapy apparatus 10 is worn on the user's head. The pivoting action of the dovetail hinge member 50 allows for pivotal, adjusted movement of the face plate 40 towards and away from the user's face to achieve the desired distance between light emitting elements of the face plate 40 and the user's facial skin according to the particular wavelength of the light that is emitted from the light elements and the particular skin condition that is being treated, as well as the shape of the user's face.

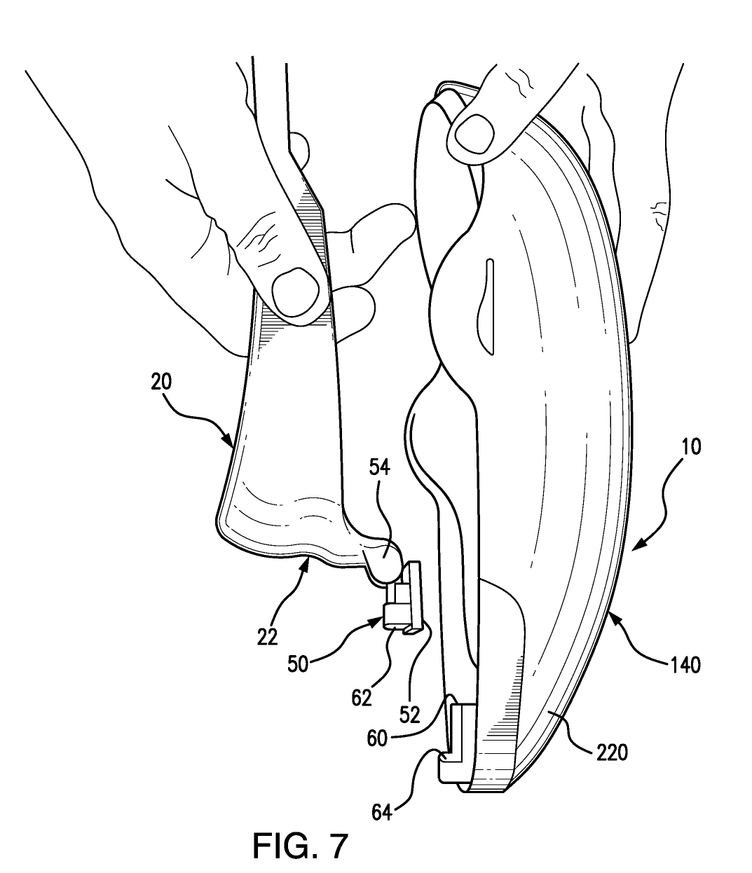

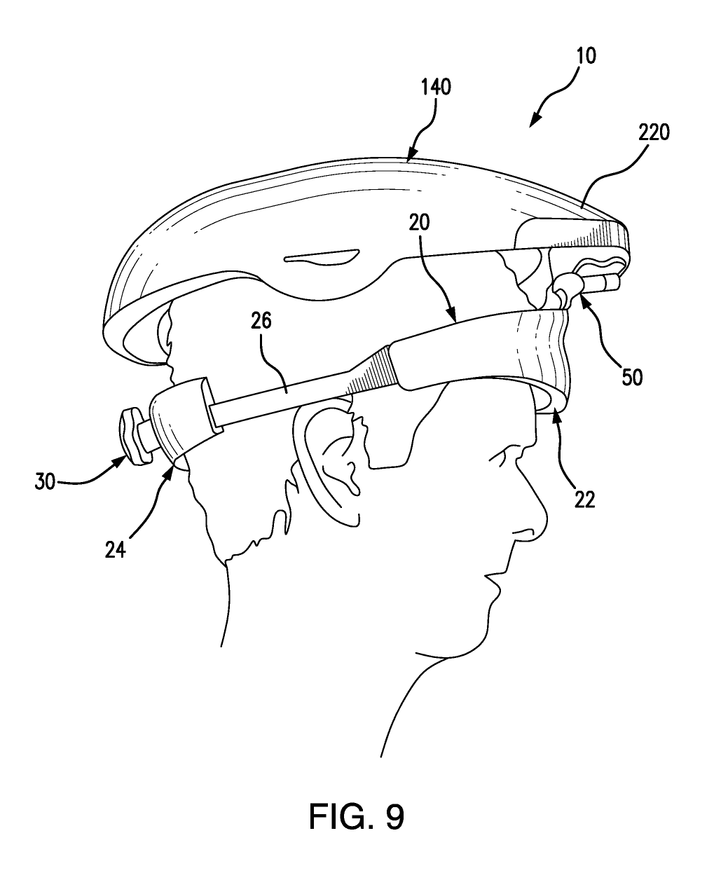

Referring to FIGS. 6 and 7, the head canopies 140 are selectively attachable and removable from the headband in the same general manner as the face plates 40 to allow interchanging of different head canopies, as well as selectively interchanging between head canopies and face plates. For instance, if the user desires to undergo a particular phototherapy treatment for the facial skin, the user would select the particular face plate 40 that has the arrangement and light colors of the light generating sources corresponding to the particular facial skin treatment desired by the user. If the user desires to undergo a particular phototherapy scalp treatment or hair growth promotion treatment, the user can then remove the face plate 40 from the headband 20, and attach a head canopy 140 having the particular arrangement and color of light emitting sources corresponding to the particular scalp or hair growth treatment desired by the user. When switching from use of a face plate 40 to use of a head canopy 140, the user simply removes the face plate 40 from the pivotal attachment member 50 and then inverts the headband 20, once removed from the user's head, so that the hinged and pivoting attachment member 50 is now on the upper side of the headband 20, as seen in FIG. 7. The pivoting attachment member 50 is then pivoted so that it is generally perpendicular to the front of the headband 20, extending outwardly therefrom, as seen in FIG. 7. In this position, the pivoting attachment member 50, and particularly the dovetail mounting member 52 is slid into and received within the slot 60 on the head canopy 140, as shown in FIGS. 7 and 9. The pivoting attachment member 50 is then pivotally operable to adjust the position of the head canopy 140 in spaced relation to the user's head. Similar to the dovetail slot 60 on the face plate 40, the dovetail slot 60 on the head canopy 140 includes magnets 64 for magnetic engagement with the corresponding magnet 62 on the pivoting mounting member 50.

Each face plate 40 and head canopy 140 in the various embodiments of the invention further includes an outer shell 220 formed of a hard plastic or other suitable material and an inner reflective shell 230 that is positioned in direct opposing relation to the user's face when the phototherapy apparatus is properly worn on the user's head during phototherapy skin treatment. Each face plate 40 and head canopy 140 of the several embodiments of the invention further includes a circuit plate that is sandwiched between the outer shell 220 and the inner reflective shell 230. Electrical power is delivered through conductors in the circuit plate to each of the light emitting elements. Electrical power may be supplied through a power cord 260 connecting to the face plate or head canopy and extending from a separate power source. An example of the power cord 260 is shown in FIGS. 3-5. Alternatively, a battery power source or other electrical power storage source may be provided onboard the phototherapy apparatus. The inner shell 230 of the head canopies may be fitted with spacer columns 240 for engaging the top of the user's head in order to set the canopy 140 at the optimal distance above the user's head, and more specifically, to ensure the optimal distance between the light emitting sources and the user's scalp.

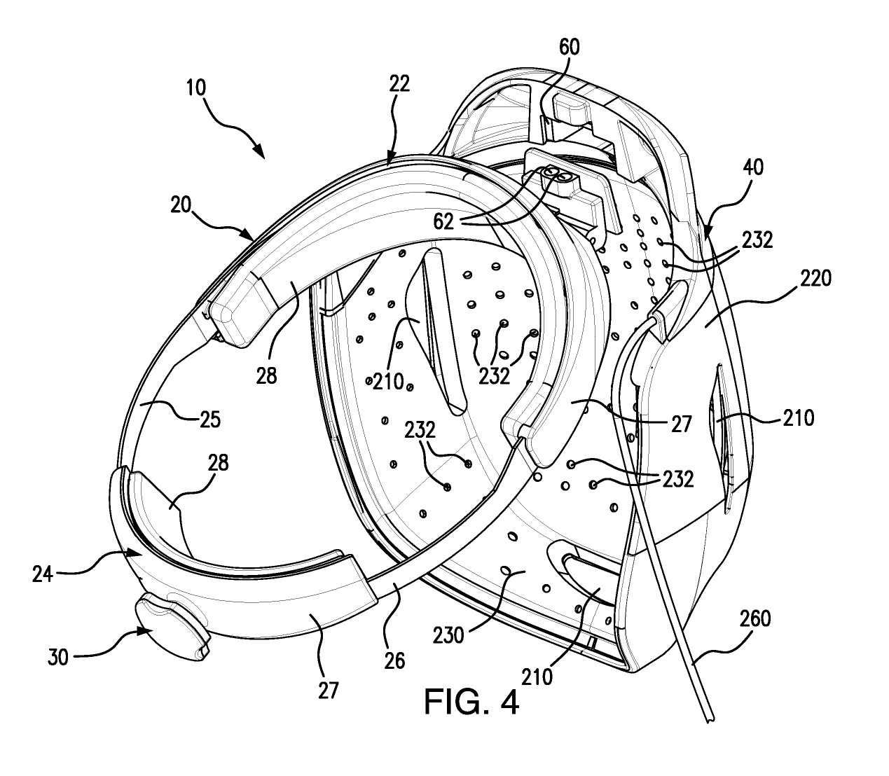

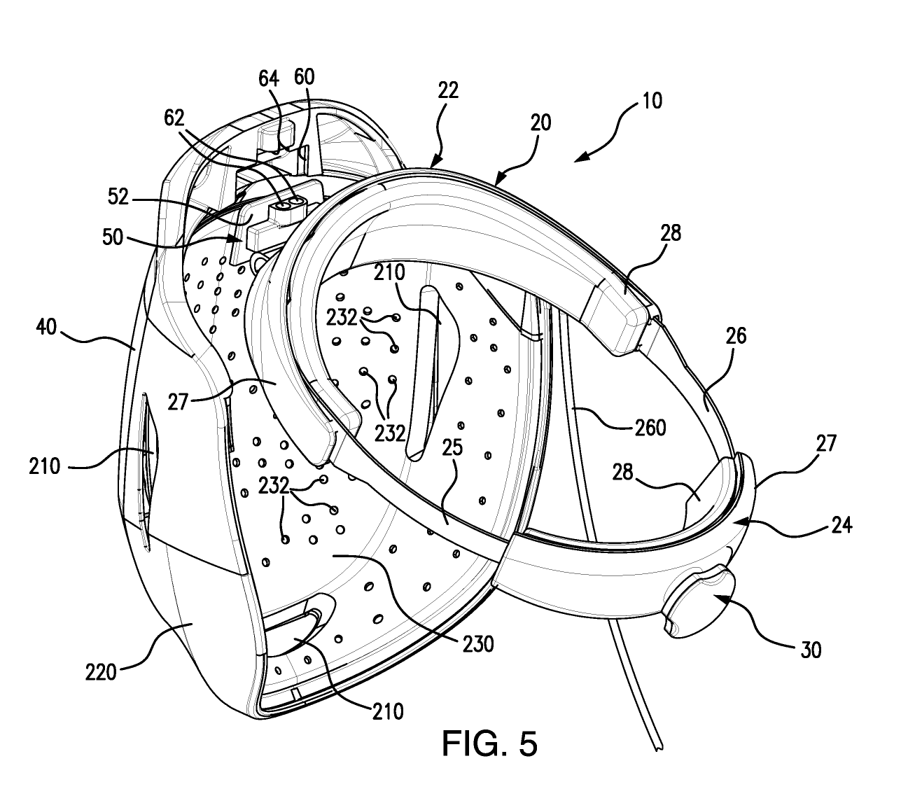

As seen in FIGS. 3-6, the inner reflective shell 230 is provided with an arrangement of holes or openings 232 that are specifically positioned for direct alignment with correspondingly positioned individual ones of the light emitting elements on the circuit plate so that light emitted from the light emitting elements passes through the holes in the reflective shell and towards the user's facial skin during use of the phototherapy apparatus. The reflective shell 230 has an exposed surface in direct opposing relation to the user's face (face plate) or scalp (head canopy) that is specifically structured to reflect light back towards the user's face or scalp. This allows light that is originally directed onto the user's face (or scalp) from the light emitting elements and which subsequently reflects off the user's face (or scalp) to be redirected onto the user's face (or scalp) for enhanced concentration of light onto the user's facial skin (or scalp) during the course of a phototherapy skin treatment. The face plates 40 are provided with vent openings 210 on the opposite sides and chin area (see FIGS. 4-5) for allowing ventilation between the face plate 40 and the user's face.

It should be noted that while the base platform component is shown throughout the drawings as a headband, it is fully contemplated within the spirit and scope of the present invention that other base platforms that are adapted to be worn on the user's head may be used such as, but not limited to, headphones, earphones, or eyeglasses, all of which would be fitted with the pivoting attachment member for attachment, removal and interchanging of the face plates and head canopies.

While the phototherapy apparatus of the present invention has been described and exemplified according to several preferred and practical embodiments thereof, it is recognized that departures from the instant disclosure are fully contemplated within the spirit and scope of the invention which is not to be limited except as defined in the following claims as interpreted under the Doctrine of Equivalents.

What is claimed is:

1. A phototherapy apparatus comprising:
a base unit structured and disposed to be worn on a user's head;
at least one face plate including a spaced array of light generating sources for emitting light within at least one wavelength range corresponding to one or more of red light, blue light, yellow light, amber light and green light;
at least one head canopy including a spaced array of light generating sources for emitting light within at least one wavelength range corresponding to one or more of red light, blue light, yellow light, amber light and green light;
at least one mounting member on the base unit for removably supporting the at least one face plate on the base unit and in spaced relation to the user's face when the base unit is worn on the user's head so that the light generating sources direct light onto the user's facial skin, and the at least one mounting member being structured and disposed for allowing adjusted positioning of the face plate in relation to the user's face to thereby adjust the distance between the array of light generating sources and the user's facial skin; and
the at least one mounting member being further structured for removably supporting the at least one head canopy on the base unit and in spaced relation to the user's scalp so that the light generating sources direct light onto the user's scalp, and the at least one mounting member being structured and disposed for allowing adjusted positioning of the at least one head canopy in relation to the user's scalp to thereby adjust the distance between the array of light generating sources and the user's scalp.

2. The phototherapy apparatus of claim 1 wherein the base unit is a headband.

3. The phototherapy apparatus of claim 2 wherein the headband includes a front portion, a rear portion and left and right strap members extending between and connecting to the front and rear portions.

4. The phototherapy apparatus of claim 3 wherein the rear portion includes a control for moving the left and right strap members relative to the rear portion to selectively enlarge or reduce a cranial circumference of the headband.

5. The phototherapy apparatus of claim 1 wherein the at least one mounting member is structured and disposed for allowing adjusted movement of the at least one face plate towards and away from the user's face and the at least one mounting member is further structured and disposed for allowing adjusted movement of the at least one head canopy towards and away from the user's scalp.

6. The phototherapy apparatus of claim 5 wherein the at least one mounting member is pivotally attached to the headband and is pivotally movable relative to the headband, and the at least one mounting member is structured and disposed for releasable engagement with the at least one face plate for removably supporting the at least one face plate on the headband and the at least one mounting member is further structured and disposed for releasable engagement with the at least one head canopy for removably supporting the at least one head canopy on the headband.

7. The phototherapy apparatus of claim 6 wherein the at least one mounting member includes a dovetail mounting structure for congruent, supporting receipt within a dovetail slot on the at least one face plate and a dovetail slot on the at least one head canopy.

8. The phototherapy apparatus of claim 7 wherein the dovetail mounting structure and the face plate have cooperating magnets for magnetically coupling the face plate to the dovetail mounting structure.

9. The phototherapy apparatus of claim 7 wherein the dovetail mounting structure and the head canopy have cooperating magnets for magnetically coupling the head canopy to the dovetail mounting structure.

10. The phototherapy apparatus as recited in claim 1 further comprising:
a plurality of face plates and a plurality of head canopies, each including a spaced array of light generating sources for emitting light within at least one wavelength range corresponding to one or more of red light, blue light, yellow light, amber light and green light, and wherein the spaced array of light generating sources on each of the plurality of face plates and each of the plurality of head canopies emits light within a different wavelength range.

11. The phototherapy apparatus as recited in claim 1 wherein the at least one face plate includes at least one vent opening therethrough for allowing ventilation between the at least one face plate and the user's face.
